(19) United States
(12) Patent Application Publication
(10) Pub. No.: US 2022/0145389 A1
(43) Pub. Date: May 12, 2022

(54) METHODS AND COMPOSITIONS USEFUL FOR NUCLEIC ACID ANALYSIS

(71) Applicant: Waters Technologies Corporation, Milford, MA (US)

(72) Inventors: Armand Gatien Ngounou Wetie, Seekonk, MA (US); Mark D. Wrona, Franklin, MA (US); Darryl W. Brousmiche, Grafton, MA (US)

(21) Appl. No.: 17/518,704

(22) Filed: Nov. 4, 2021

Related U.S. Application Data
(60) Provisional application No. 63/110,668, filed on Nov. 6, 2020.

(51) Int. Cl. C12Q 1/6876; C12Q 1/6825

(52) U.S. Cl. CPC C12Q 1/6876; C12Q 1/6825

(57) ABSTRACT

The present disclosure relates to compositions that comprise a fluorescent and/or MS-active nucleic acid probe that comprises (a) a nucleic-acid-based moiety and (b) a fluorescent moiety comprising a fluorophore, an MS-active moiety, or both a fluorescent moiety and an MS-active moiety, linked to the nucleic-acid-based moiety, among other aspects.

### CROSS-REFERENCE TO RELATED APPLICATIONS

This application claims the benefit of and priority to U.S. Provisional Patent Application No. 63/110,668, filed on Nov. 6, 2020, the entire contents of which is hereby incorporated by reference.

### FIELD

The present disclosure relates to compositions and methods that may be used, for example, in conjunction with the processing and/or analysis of nucleic acids. In various embodiments, the present disclosure relates to compositions and methods that may be used for the processing and/or analyses of nucleic acids in a biological or non-biological sample of interest.

### BACKGROUND

Nucleic acid analysis is important in a number of areas including pharmaceuticals, biopharmaceuticals, clinical settings, and food science, among other areas. The present disclosure provides methods and compositions for nucleic acid analysis that are based on fluorescent, ultraviolet (UV) and/or mass spectrometry (MS) analyses, potentially coupled with chromatographic and/or affinity-ligand-based separation/purification techniques. Through the enhanced sensitivity associated with fluorescence detection, the enhanced selectivity associated with MS analysis, or combination of both, the methods and compositions herein provide potential for confident quantitation, identification and characterization of nucleic acids.

### SUMMARY

In some aspects, the present disclosure is directed to compositions that comprise a fluorescent and/or MS-active nucleic acid probe that comprises (a) a nucleic-acid-based moiety and (b) a fluorescent moiety comprising a fluorophore, an MS-active moiety, or both a fluorescent moiety and an MS-active moiety, linked to the nucleic-acid-based moiety. Thus, in some embodiments, the fluorescent and/or MS-active nucleic acid probe comprises a fluorescent moiety but not an MS-active moiety. In some embodiments, the fluorescent and/or MS-active nucleic acid probe comprises an MS-active moiety but not a fluorescent moiety. In some embodiments, the fluorescent and/or MS-active nucleic acid probe comprises both a fluorescent moiety comprising and a MS-active moiety.

In some embodiments, which can be used in conjunction with any of the above aspects and embodiments, the nucleic-acid-based moiety comprises a nucleic acid or a nucleic acid derivative or a nucleic acid analog. For example, the nucleic-acid-based moiety may comprise a deoxyribonucleic acid (DNA), a ribonucleic acid (RNA), a peptide nucleic acid (PNA), or a mixed nucleic acid, among others.

In some embodiments, which can be used in conjunction with any of the above aspects and embodiments, the fluorescent and/or MS-active nucleic acid probe further comprises an affinity ligand.

In some embodiments, which can be used in conjunction with any of the above aspects and embodiments, the compositions further comprise a fluorescent and/or MS-active hybridized moiety in which a target nucleic acid molecule hybridized to the nucleic-acid-based moiety.

In some embodiments, which can be used in conjunction with any of the above aspects and embodiments, the compositions comprise a plurality of fluorescent and/or MS-active nucleic acid probes having a plurality of nucleic-acid-based moieties,

For example, (a) the compositions may comprise first and second fluorescent and/or MS-active nucleic acid probes having first and second nucleic-acid-based moieties, (b) the compositions may comprise first, second and third fluorescent and/or MS-active nucleic acid probes having first, second and third nucleic-acid-based moieties, (c) the compositions may comprise first, second, third and fourth fluorescent and/or MS-active nucleic acid probes having first, second, third and fourth nucleic-acid-based moieties, (d) and so forth. In some embodiments, (a) the compositions may comprise first and second fluorescent and/or MS-active hybridized moieties, (b) the compositions may comprise first, second and third fluorescent and/or MS-active hybridized moieties, (c) the compositions may comprise first, second, third and fourth fluorescent and/or MS-active hybridized moieties, (d) and so forth. In these embodiments, the first fluorescent and/or MS-active nucleic acid probe may comprise a first fluorescent moiety that fluoresces at a first wavelength, the second fluorescent and/or MS-active nucleic acid probe may comprise a second fluorescent moiety that fluoresces at a second wavelength that is different from the first wavelength, the third fluorescent and/or MS-active nucleic acid probe may comprise a third fluorescent moiety that fluoresces at a third wavelength that is different from the first and second wavelengths, the fourth fluorescent and/or MS-active nucleic acid probe may comprise a fourth fluorescent moiety that fluoresces at a fourth wavelength that is different from the first, second and third wavelengths, and so forth.

In other aspects, the present disclosure is directed methods for detecting a target nucleic acid molecule in a sample containing or suspected of containing the target nucleic acid molecule in which the method comprises (a) contacting the sample with a composition comprising a fluorescent and/or MS-active nucleic acid probe in accordance with any of the above aspects and embodiments, wherein the composition is contacted with the sample under conditions that allow the nucleic-acid-based moiety of the fluorescent and/or MS-active nucleic acid probe to hybridize with the target nucleic acid molecule and form a modified sample containing a fluorescent and/or MS-active hybridized moiety; and (c) analyzing the modified sample containing the fluorescent and/or MS-active hybridized moiety using mass spectrometry, using fluorescence spectroscopy, or using both mass spectrometry and fluorescence spectroscopy.

In embodiments where the modified sample containing the fluorescent and/or MS-active hybridized moiety is analyzed using mass spectrometry, the mass spectrometry may be performed in positive ion mode and the MS-active moiety may provide the fluorescent and/or MS-active hybridized moiety with an increased positive charge, or the mass spectrometry may be performed in negative ion mode and the MS-active moiety may provide the fluorescent and/or MS-active hybridized moiety with an increased negative charge.

In some embodiments, which can be used in conjunction with any of the above aspects and embodiments, the nucleic-acid-based moiety contains five or more consecutive nucleotides that are complementary to a sequence of five or more consecutive nucleotides of the target nucleic acid molecule.

In some embodiments, which can be used in conjunction with any of the above aspects and embodiments, the target nucleic acid molecule comprises RNA, DNA or a mixed oligonucleotide.

In some embodiments, which can be used in conjunction with any of the above aspects and embodiments, the target nucleic acid molecule is selected from complementary DNA (cDNA), mitochondrial DNA (mDNA), messenger RNA (mRNA), short interfering RNA (siRNA), transfer RNA (tRNA), ribosomal RNA (rRNA), microRNA (miRNA), small nuclear RNA (snRNA), or complementary RNA (cRNA).

In some embodiments, which can be used in conjunction with any of the above aspects and embodiments, the composition comprises a plurality of fluorescent and/or MS-active nucleic acid probes having a plurality of nucleic-acid-based moieties, and the modified sample comprises a plurality fluorescent and/or MS-active hybridized moieties.

For example, (a) the composition may comprise first and second fluorescent and/or MS-active nucleic acid probes having first and second nucleic-acid-based moieties, and the modified sample may comprise first and second fluorescent and/or MS-active hybridized moieties, (b) the composition may comprise first, second and third fluorescent and/or MS-active nucleic acid probes having first, second and third nucleic-acid-based moieties, and the modified sample may comprise first, second and third fluorescent and/or MS-active hybridized moieties, (c) the composition may comprise first, second, third and fourth fluorescent and/or MS-active nucleic acid probes having first, second, third and fourth nucleic-acid-based moieties, and the modified sample may comprise first, second, third and fourth fluorescent and/or MS-active hybridized moieties, (d) and so forth. In these embodiments, the first fluorescent and/or MS-active nucleic acid probe may comprise a first fluorescent moiety that fluoresces at a first wavelength, the second fluorescent and/or MS-active nucleic acid probe may comprise a second fluorescent moiety that fluoresces at a second wavelength that is different from the first wavelength, the third fluorescent and/or MS-active nucleic acid probe may comprise a third fluorescent moiety that fluoresces at a third wavelength that is different from the first and second wavelengths, the fourth fluorescent and/or MS-active nucleic acid probe may comprise a fourth fluorescent moiety that fluoresces at a fourth wavelength that is different from the first, second and third wavelengths, and so forth.

In some embodiments, which can be used in conjunction with any of the above aspects and embodiments, the fluorescent and/or MS-active nucleic acid probe(s) may further comprise(s) an affinity ligand and the method may further comprise contacting the modified sample with a substrate having a surface that comprises a bound surface species that interacts with the affinity ligand, such that the hybridized moiety is captured by the substrate.

In some embodiments, which can be used in conjunction with any of the above aspects and embodiments, the method comprises performing liquid chromatography on the modified sample thereby separating the fluorescent and/or MS-active hybridized moiety prior to analyzing the modified sample using mass spectrometry, using fluorescence spectroscopy, or using both mass spectrometry and fluorescence spectroscopy. For example, the liquid chromatography method may be selected from reversed phase chromatography, mixed mode chromatography, ion-pair chromatography, ion-exchange chromatography, hydrophilic interaction liquid chromatography (HILIC), hydrophobic interaction chromatography (HIC), and capillary electrophoresis (CE), among others.

In other aspects, the present disclosure is directed methods for detecting a target nucleic acid molecule in a sample containing or suspected of containing the target nucleic acid molecule in which the method comprises (a) contacting the sample with a functional-group-containing nucleic acid probe that comprises a nucleic-acid-based moiety and a functional group such as a primary or secondary amine, a carboxylic acid group, a thiol group, or an alcohol group, under conditions that allow the nucleic-acid-based moiety of the functional-group-containing nucleic acid probes to hybridize with the target nucleic acid molecule and form a sample that contains a hybridized moiety; (b) contacting the sample that contains the hybridized moiety with a fluorescent and/or MS-active tagging compound that comprises (i) a fluorescent moiety comprising a fluorophore, an MS-active moiety, or both a fluorescent moiety comprising a fluorophore and an MS-active moiety and (ii) a reactive moiety that is reactive with the functional group of the functional-group-containing nucleic acid probe, thereby forming a modified sample that contains a fluorescent and/or MS-active hybridized moiety; and (c) analyzing the sample that contains the fluorescent and/or MS-active hybridized moiety using mass spectrometry, using fluorescence spectroscopy, or using both mass spectrometry and fluorescence spectroscopy.

In some embodiments, which can be used in conjunction with any of the above aspects and embodiments, the target nucleic acid molecule comprises RNA, DNA or a mixed oligonucleotide.

In some embodiments, which can be used in conjunction with any of the above aspects and embodiments, the target nucleic acid molecule is selected from complementary DNA (cDNA), mitochondrial DNA (mDNA), messenger RNA (mRNA), short interfering RNA (siRNA), transfer RNA (tRNA), ribosomal RNA (rRNA), microRNA (miRNA), small nuclear RNA (snRNA), or complementary RNA (cRNA).

In some embodiments, which can be used in conjunction with any of the above aspects and embodiments, the functional-group-containing nucleic acid probe contains five or more consecutive nucleotides that are complementary to a sequence of five or more consecutive nucleotides of the target nucleic acid molecule.

In some embodiments, which can be used in conjunction with any of the above aspects and embodiments, the functional-group-containing nucleic acid probe comprises PNA.

In some embodiments, which can be used in conjunction with any of the above aspects and embodiments, the reactive moiety is selected from a succinimidyl carbamate group, a succinimidyl ester group, or an isocyanate group.

In some embodiments, which can be used in conjunction with any of the above aspects and embodiments, the fluorescent and/or MS-active tagging compound comprises both the fluorescent moiety and the MS-active moiety and the sample that contains the fluorescent and/or MS-active hybridized moiety is analyzed using both mass spectrometry and fluorescence spectroscopy.

In some embodiments, which can be used in conjunction with any of the above aspects and embodiments, the fluorescent and/or MS-active tagging compound is selected

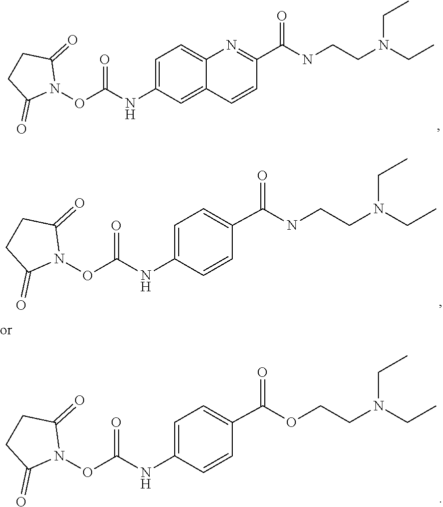 [SMILES: CCN(CC)CCNC(=O)c1ccc2cc(NC(=O)ON3C(=O)CCC3=O)ccc2n1 ; CCN(CC)CCNC(=O)c1ccc(NC(=O)ON2C(=O)CCC2=O)cc1 ; CCN(CC)CCOC(=O)c1ccc(NC(=O)ON2C(=O)CCC2=O)cc1]

In some embodiments, which can be used in conjunction with any of the above aspects and embodiments, the fluorescent and/or MS-active tagging compound further comprises an affinity ligand and the method further comprises contacting the sample that contains the fluorescent and/or MS-active hybridized moiety with a substrate that comprises a bound surface species that interacts with the affinity ligand such that the fluorescent and/or MS-active tagging compound is captured by the substrate.

In some embodiments, which can be used in conjunction with any of the above aspects and embodiments, the method further comprises performing liquid chromatography on the sample that contains the fluorescent and/or MS-active hybridized moiety thereby separating the fluorescent and/or MS-active hybridized moiety prior to analysis using mass spectrometry, using fluorescence spectroscopy, or using both mass spectrometry and fluorescence spectroscopy.

In other aspects, the present disclosure is directed methods of making a fluorescent and/or MS-active nucleic acid probes that comprise reacting (a) a functional-group-containing nucleic acid that comprises a nucleic-acid-based moiety and a primary or secondary amine with (b) a fluorescent and/or MS-active tagging compound that comprises (i) a fluorescent moiety comprising a fluorophore, an MS-active moiety, or both a fluorescent moiety comprising a fluorophore and an MS-active moiety and (ii) a reactive moiety that is reactive with the functional group of the functional-group-containing nucleic acid probe, thereby forming the fluorescent and/or MS-active nucleic acid probe.

In some embodiments, which can be used in conjunction with any of the above aspects and embodiments, the nucleic-acid-based moiety comprises a peptide nucleic acid (PNA).

In some embodiments, which can be used in conjunction with any of the above aspects and embodiments, the reactive moiety is selected from a succinimidyl carbamate group, a succinimidyl ester group, or an isocyanate group.

In some embodiments, which can be used in conjunction with any of the above aspects and embodiments, the fluorescent and/or MS-active tagging compound comprises both the fluorescent moiety and the MS-active moiety.

In some embodiments, which can be used in conjunction with any of the above aspects and embodiments, the reactive compound comprises

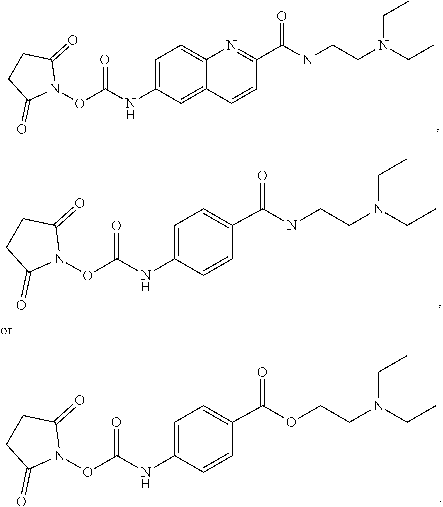 [SMILES: CCN(CC)CCNC(=O)c1ccc2cc(NC(=O)ON3C(=O)CCC3=O)ccc2n1 ; CCN(CC)CCNC(=O)c1ccc(NC(=O)ON2C(=O)CCC2=O)cc1 ; CCN(CC)CCOC(=O)c1ccc(NC(=O)ON2C(=O)CCC2=O)cc1]

In other aspects, the present disclosure is directed to kits that comprise (1) a first fluorescent and/or MS-active nucleic acid probe that comprises (a) a first nucleic-acid-based moiety and (b) a fluorescent moiety comprising a fluorophore, an MS-active moiety, or both a fluorescent moiety and an MS-active moiety, linked to the first nucleic-acid-based moiety and (2) a second fluorescent and/or MS-active nucleic acid probe that comprises (a) a second nucleic-acid-based moiety and (b) a fluorescent moiety comprising a fluorophore, an MS-active moiety, or both a fluorescent moiety and an MS-active moiety, linked to the second nucleic-acid-based moiety.

In some embodiments, the kits may comprise, a third fluorescent and/or MS-active nucleic acid probe that comprises (a) a third nucleic-acid-based moiety and (b) a fluorescent moiety comprising a fluorophore, an MS-active moiety, or both a fluorescent moiety and an MS-active moiety, linked to the third nucleic-acid-based moiety, and so forth.

In some embodiments, which can be used in conjunction with any of the above aspects and embodiments, the fluorescent and/or MS-active nucleic acid probes comprise the fluorescent moiety.

In some embodiments, which can be used in conjunction with any of the above aspects and embodiments, the fluorescent and/or MS-active nucleic acid probes comprise the MS-active moiety.

In some embodiments, which can be used in conjunction with any of the above aspects and embodiments, the fluorescent and/or MS-active nucleic acid probes comprise both the fluorescent moiety comprising and the MS-active moiety.

In some embodiments, which can be used in conjunction with any of the above aspects and embodiments, the nucleic-acid-based moieties are nucleic acids, nucleic acid derivatives, or nucleic acid analogs.

In some embodiments, which can be used in conjunction with any of the above aspects and embodiments, the nucleic-acid-based moieties comprise deoxyribonucleic acid (DNA), ribonucleic acid (RNA), a peptide nucleic acid (PNA), or mixed nucleic acids.

In some embodiments, which can be used in conjunction with any of the above aspects and embodiments, the fluorescent and/or MS-active nucleic acid probes further comprises an affinity ligand.

In some embodiments, which can be used in conjunction with any of the above aspects and embodiments, the first fluorescent and/or MS-active nucleic acid probe comprises a first fluorescent moiety that fluoresces at a first wavelength, the second fluorescent and/or MS-active nucleic acid probe comprises a second fluorescent moiety that fluoresces at a second wavelength that is different from the first wavelength, the third fluorescent and/or MS-active nucleic acid probe, if present, comprises a third fluorescent moiety that fluoresces at a third wavelength that is different from the first and second wavelengths, and so forth.

It is noted that, in any of the aspects and embodiments described herein, the fluorescent moiety may be replaced with a UV-absorbing moiety and the UV-absorbing moiety may be analyzed using UV spectroscopy.

### DETAILED DESCRIPTION

In various aspects, the present disclosure pertains to compositions and methods for detecting one or more target nucleic acid molecules in a sample containing or suspected of containing the one or more target nucleic acid molecules.

In some embodiments, the present disclosure pertains to fluorescent and/or MS-active nucleic acid probes that, which comprise (i) a nucleic-acid-based moiety linked to (ii) a fluorescent moiety comprising a fluorophore, an MS-active moiety, or both a fluorescent moiety comprising a fluorophore and an MS-active moiety linked.

In some embodiments, the present disclosure pertains to methods that use such fluorescent and/or MS-active nucleic acid probes for detecting one or more target nucleic acid molecules in a sample that contains or is suspected of containing the one or more target nucleic acid molecules. These methods comprise: (a) contacting the sample with one or more of such fluorescent and/or MS-active nucleic acid probes under conditions that allow the nucleic-acid-based moieties of the one or more fluorescent and/or MS-active nucleic acid probes to hybridize with the one or more target nucleic acid molecules and form a sample that contains one or more fluorescent and/or MS-active hybridized moieties; and (b) analyzing the sample that contains the one or more fluorescent and/or MS-active hybridized moieties using mass spectrometry, using fluorescence spectroscopy, or using both mass spectrometry and fluorescence spectroscopy.

In some embodiments, the present disclosure pertains to methods for detecting one or more target nucleic acid molecules in a sample that contains or is suspected of containing the one or more target nucleic acid molecules. These methods comprise: (a) contacting the sample with one or more-functional-group-containing nucleic acid probes that comprise a nucleic-acid-based moiety and a functional group such as a primary or secondary amine, a carboxylic acid group, a thiol group, or an alcohol group, among others, under conditions that allow the nucleic-acid-based moiety of the one or more functional-group-containing nucleic acid probes to hybridize with the one or more target nucleic acid molecules and form a sample that contains one or more hybridized moieties; (b) contacting the hybridized moiety with a fluorescent and/or MS-active tagging compound that comprises (i) a fluorescent moiety comprising a fluorophore, an MS-active moiety, or both a fluorescent moiety comprising a fluorophore and an MS-active moiety and (ii) a reactive moiety that is reactive with the functional group of the one or more functional-group-containing nucleic acid probes, thereby forming a sample that contains one or more fluorescent and/or MS-active hybridized moieties, and (c) analyzing the sample that contains the one or more fluorescent and/or MS-active hybridized moieties using mass spectrometry, using fluorescence spectroscopy, or using both mass spectrometry and fluorescence spectroscopy.

Potential samples that can be analyzed using the above methods include various biological and non-biological samples, including biological fluids such as plasma, serum, whole blood, oral fluids, and urine, animal tissue, plant tissue, fungi, microorganisms, cell culture, formulations, synthetic process products, certain foods, and environmental samples, among others.

Any suitable nucleic-acid-based moiety may be used in the above fluorescent and/or MS-active nucleic acid probes or in the above amine-containing nucleic acid probes. In particular embodiments, the nucleic-acid-based moiety is a deoxyribonucleic acid (DNA), a ribonucleic acid (RNA), a peptide nucleic acid (PNA), nucleic acid analog, or a mixed nucleic acid. In various embodiments, the nucleic-acid-based moiety contains five or more consecutive nucleotides that are complementary to a sequence of five or more nucleotides of the target nucleic acid molecule, for example, up to 100 nucleotides of the target nucleic acid molecule. Thus the nucleic-acid-based moiety contain from 5 to 10 to 25 to 50 to 75 to 100 consecutive nucleotides may that are complementary to a sequence of the target nucleic acid molecule that ranges from 5 to 10 to 25 to 50 to 75 to 100 nucleotides, respectively.

Any target nucleic acid molecule may be analyzed using the methods described herein, including RNA target nucleic acid molecules, DNA target nucleic acid molecules, mixed oligonucleotide target nucleic acid molecules. Particular examples of such target nucleic acid molecules include complementary DNA (cDNA), mitochondrial DNA (mDNA), messenger RNA (mRNA), short interfering RNA (siRNA), transfer RNA (tRNA), ribosomal RNA (rRNA), microRNA (miRNA), small nuclear RNA (snRNA), complementary RNA (cRNA), and ribozymes, among others.

Any suitable fluorescent moiety or any suitable plurality of fluorescent of moieties (for example, a first fluorescent moiety having a first emission maximum, a second fluorescent moiety having a second emission maximum differing from the first emission maximum, a third fluorescent moiety having a third emission maximum differing from the first and second emission maxima, etc.) can be used in the above fluorescent and/or MS-active nucleic acid probes or the above fluorescent and/or MS-active tagging compounds. Fluorescent moieties include those fluorescent moieties described in U.S. Patent Pub. No. 2014/0242709, U.S. Patent Pub. No. 2014/0350263, U.S. Patent Pub. No. 2016/0139136, U.S. Pat. Nos. 10,436,790, 10,416,166, the disclosures of which are incorporated by reference. Particular beneficial fluorescent moieties include those that comprise substituted and unsubstituted aromatic and heteroaromatic groups such as a substituted or unsubstituted phenyl group, a naphthalene group, a quinoline group, a substituted or unsubstituted coumarin family group, a substituted or unsubstituted rhodamine family group, a substituted or unsubstituted oxazine family group, a substituted or unsubstituted carbopyronine family group, a substituted or unsubstituted anthracene group, a substituted or unsubstituted phenanthrene group, a substituted or unsubstituted pyrene group, a substituted or unsubstituted cyanine family group, a substituted or unsubstituted fluorescein family group, or a substituted or unsubstituted carbazole group, among others.

In some embodiments two or more fluorescent nucleic acid probes (which may also be MS-active) may be used, each having different a fluorescent moiety that fluoresces at a different wavelength and a different nucleic-acid-based moiety for binding to a different target nucleic acid molecule. For example, the two or more of the following may be used: (a) a first fluorescent nucleic acid probe (which may also be MS-active) having a first fluorescent moiety that fluoresces at a first wavelength and a first nucleic-acid-based moiety for binding to a first target nucleic acid molecule, (b) a second fluorescent nucleic acid probe (which may also be MS-active) having a second a fluorescent moiety that fluoresces at a second wavelength differing from the first wavelength and a second nucleic-acid-based moiety differing from the first nucleic-acid-based moiety for binding to a second target nucleic acid molecule differing from the first target nucleic acid molecule, (c) a third fluorescent nucleic acid probe (which may also be MS-active) having a third a fluorescent moiety that fluoresces at a third wavelength differing from the first and second wavelengths and a third nucleic-acid-based moiety differing from the first and second nucleic-acid-based moieties for binding to a third target nucleic acid differing from the first and second target nucleic acid molecules, (d) a fourth fluorescent nucleic acid probe (which may also be MS-active) having a fourth a fluorescent moiety that fluoresces at a fourth wavelength differing from the first, second and third wavelengths and a fourth nucleic-acid-based moiety differing from the first, second and third nucleic-acid-based moieties for binding to a fourth target nucleic acid molecule differing from the first, second and third target nucleic acid molecules, (e) and so forth.

Any suitable MS-active moiety can be used in the above fluorescent and/or MS-active nucleic acid probes and the above fluorescent and/or MS-active tagging compounds. MS active moieties include those fluorescent moieties described in U.S. Patent Pub. No. 2014/0242709, U.S. Patent Pub. No. 2014/0350263, U.S. Patent Pub. No. 2016/0139136, U.S. Pat. Nos. 10,436,790, and 10,416,166. Particular beneficial MS-active moieties include those that comprise amine-containing moieties including tertiary-amine-containing moieties, phosphonic-acid-containing or phosphonate-containing moieties, and sulfonic-acid-containing or sulfonate-containing moieties, among others. In some embodiments, the MS-active moiety provides an increased positive charge during mass spectrometry analysis (which is useful, for example, in conjunction with positive ion mode mass spectrometry). In some embodiments, the MS-active moiety provides an increased negative charge during mass spectrometry analysis (which is useful, for example, in conjunction with negative ion mode ion mode mass spectrometry).

Any reactive moiety that is reactive with a primary or secondary amine can be used with the above fluorescent and/or MS-active tagging compounds. Reactive moieties include those reactive moieties described in U.S. Patent Pub. No. 2014/0242709, U.S. Patent Pub. No. 2014/0350263, U.S. Patent Pub. No. 2016/0139136, U.S. Pat. Nos. 10,436,790, and 10,416,166. Particular beneficial reactive moieties include a succinimidyl carbamate group, a succinimidyl ester group, or an isocyanate group, among others. Such reactive moieties can be reacted, for example, with one or more primary or secondary amines of an amine-containing nucleic acid probe, thereby forming a fluorescent and/or MS-active nucleic acid probe. A succinimidyl carbamate group reacts, for example, with a primary amine to form a urea linkage, a succinimidyl ester group reacts, for example, with a primary amine to form an amide linkage, and an isocyanate group reacts, for example, with a primary amine to form urea linkage succinimidyl carbamate.

Particular tagging compounds for use in the present disclosure include AccQ-Fluor™,

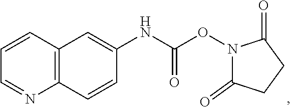 [SMILES: O=C(Nc1ccc2ncccc2c1)ON1C(=O)CCC1=O]

a fluorescent tagging compound available from Waters Corporation, Rapifluor-MS®,

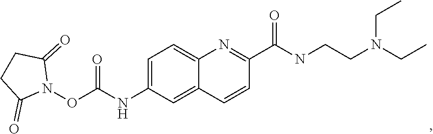 [SMILES: CCN(CC)CCNC(=O)c1ccc2cc(NC(=O)ON3C(=O)CCC3=O)ccc2n1]

a fluorescent and MS-active tagging compound available from Waters Corporation, InstantPC™

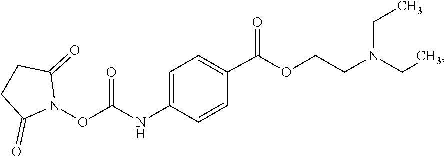 [SMILES: CCN(CC)CCOC(=O)c1ccc(NC(=O)ON2C(=O)CCC2=O)cc1]

a fluorescent and MS-active tagging compound available from ProZyme, Inc., or

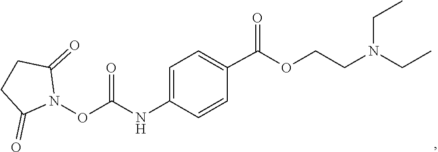 [SMILES: CCN(CC)CCOC(=O)c1ccc(NC(=O)ON2C(=O)CCC2=O)cc1]

a fluorescent and MS-active tagging compound.

Other aspects of the present disclosure pertain to methods of making a fluorescent and/or MS-active nucleic acid probe. The methods comprise reacting (a) an amine-containing nucleic acid that comprises a nucleic-acid-based moiety and a primary or secondary amine with (b) a fluorescent and/or MS-active tagging compound that comprises (i) a fluorescent moiety comprising a fluorophore, an MS-active moiety, or both a fluorescent moiety comprising a fluorophore and an MS-active moiety and (ii) a reactive moiety that is reactive with the primary or secondary amine of the one or more amine-containing nucleic acid probe, thereby forming the fluorescent and/or MS-active nucleic acid probe. For example, the amine-containing nucleic acid and the fluorescent and/or MS-active tagging compound may be selected from those described above.

In some embodiments, the fluorescent and/or MS-active nucleic acid probes described herein, or the fluorescent and/or MS-active tagging compounds described herein will further comprise an affinity ligand or aptamer (e.g., biotin, digoxigenin, dinitrophenol, human serum albumin (HSA) aptamer, immunoglobulin G (IgG) Fc aptamer, fibrinogen aptamer, etc.) that binds to a given target protein (e.g. avidin, streptavidin, anti-digoxigenin-antibody, anti-dinitrophenol-antibody, HSA, IgG, fibrinogen, etc.). In such cases, the resulting fluorescent and/or MS-active hybridized moiety will contain an affinity ligand or aptamer (e.g., biotin, digoxigenin, dinitrophenol, HSA aptamer immunoglobulin IgG aptamer, fibrinogen aptamer, etc.), allowing the fluorescent and/or MS-active hybridized moiety to be captured by a solid substrate (e.g., beads, plates, etc.) having a surface that comprises a bound surface species that interacts with the affinity ligand, for example, a bound target protein that interacts with the affinity ligand (e.g. avidin, streptavidin, anti-digoxigenin-antibody, anti-dinitrophenol-antibody, HSA, IgG, fibrinogen, etc.).

In the above described methods, once formed, the fluorescent and/or MS-active hybridized moiety can be analyzed using mass spectrometry, using fluorescence spectroscopy, or using both mass spectrometry and fluorescence spectroscopy.

Prior performing such analyses, if desired, the sample that contains the one or more fluorescent and/or MS-active hybridized moieties can be subjected to one or more suitable separation techniques.

For example, the sample that contains the one or more fluorescent and/or MS-active hybridized moieties can be separated using one or more suitable chromatographic techniques, thereby purifying the hybridized moieties prior to detecting the hybridized moieties.

Examples of suitable chromatographic techniques include liquid chromatography techniques such as reversed phase chromatography, mixed mode chromatography, ion-pair chromatography, ion-exchange chromatography, hydrophilic interaction liquid chromatography (HILIC), hydrophobic interaction chromatography (HIC), and capillary electrophoresis (CE).

As another example, in embodiments where the resulting fluorescent and/or MS-active hybridized moieties contain an affinity ligand or aptamer, the sample that contains the one or more fluorescent and/or MS-active hybridized moieties can be immobilized on a solid substrate having a surface that comprises a bound surface species that interacts with the affinity ligand as described above. Subsequently, the fluorescent and/or MS-active hybridized moiety may be released from the solid substrate for further separation or analysis.

After any desired separation technique is performed on the sample that contains the one or more fluorescent and/or MS-active hybridized moieties (e.g., chromatography, affinity separation, etc.), the one or more fluorescent and/or MS-active hybridized moieties in the sample can be using mass spectrometry, using fluorescence spectroscopy, or using both mass spectrometry and fluorescence spectroscopy. For example, an eluent stream from the chromatographic separation process may be analyzed using mass spectrometry, using fluorescence spectroscopy, or using both mass spectrometry and fluorescence spectroscopy

Where the one or more hybridized moieties are both fluorescent and/or MS-active, a system can be employed wherein, upon detection of a fluorescent signal, the portion of the sample exhibiting the fluorescent signal can be shunted to a mass spectrometer for MS analysis.

As noted above, in some embodiments, two or more fluorescent nucleic acid probes (which may also be MS-active) may be used, each having different a fluorescent moiety that fluoresces at different wavelength and a different nucleic-acid-based moiety for binding to a different target nucleic acid molecule. In such embodiments, portions of the sample exhibiting fluorescence at particular wavelengths can be separately shunted to a mass spectrometer for MS analysis.

Examples of mass spectrometry which may be employed include tandem mass spectrometry (MS/MS), electrospray ionization mass spectrometry (ESI-MS), matrix-assisted laser desorption/ionization mass spectrometry (MALDI-MS), and time-of-flight mass spectrometry (TOFMS), among others. Examples of fluorescence spectrometry include, for example, filter fluorometers, which use filters to isolate incident light (from an excitation source) and fluorescent light (from the sample), and spectrofluorometers, which use diffraction grating monochromators to isolate the incident light and fluorescent light.

1. A composition comprising a fluorescent and/or MS-active nucleic acid probe that comprises (a) a nucleic-acid-based moiety and (b) a fluorescent moiety comprising a fluorophore, an MS-active moiety, or both a fluorescent moiety and an MS-active moiety, linked to the nucleic-acid-based moiety.

2. The composition of claim 1, wherein the fluorescent and/or MS-active nucleic acid probe comprises the fluorescent moiety.

3. The composition of claim 1, wherein the fluorescent and/or MS-active nucleic acid probe comprises the MS-active moiety.

4. The composition of claim 1, wherein the fluorescent and/or MS-active nucleic acid probe comprises both the fluorescent moiety comprising and the MS-active moiety.

5. The composition of any of claim 1, wherein the nucleic-acid-based moiety comprises a nucleic acid, a nucleic acid derivative or a nucleic acid analog.

6. The composition of any of claim 1, wherein the nucleic-acid-based moiety comprises a deoxyribonucleic acid (DNA), a ribonucleic acid (RNA), a peptide nucleic acid (PNA), or a mixed nucleic acid.

7. The composition of any of claim 1, wherein the fluorescent and/or MS-active nucleic acid probe further comprises an affinity ligand.

8. The composition of any of claim 1, wherein the composition comprises first and second fluorescent and/or MS-active nucleic acid probes having first and second nucleic-acid-based moieties.

9. The composition of claim 8, wherein the first fluorescent and/or MS-active nucleic acid probe comprises a first fluorescent moiety that fluoresces at a first wavelength and wherein the second fluorescent and/or MS-active nucleic acid probe comprises a second fluorescent moiety that fluoresces at a second wavelength that is different from the first wavelength.

10. The composition of any of claim 1, further comprising a target nucleic acid molecule hybridized to the nucleic-acid-based moiety.

11. The composition of claim 10, wherein the nucleic-acid-based moiety contains five or more consecutive nucleotides that are complementary to a sequence of five or more consecutive nucleotides of the target nucleic acid molecule.

12. The composition of claim 10, wherein the target nucleic acid molecule comprises RNA, DNA, a nucleic acid analog, or a mixed oligonucleotide.

13. The composition of claim 10, wherein the target nucleic acid molecule is selected from complementary DNA (cDNA), mitochondrial DNA (mDNA), messenger RNA (mRNA), short interfering RNA (siRNA), transfer RNA (tRNA), ribosomal RNA (rRNA), microRNA (miRNA), small nuclear RNA (snRNA), or complementary RNA (cRNA).

14. The composition of any of claim 9, wherein the composition comprises first and second fluorescent and/or MS-active nucleic acid probes having first and second nucleic-acid-based moieties, and wherein the composition comprises first and second target nucleic acid molecules hybridized to the first and second nucleic-acid-based moieties.

15. The composition of claim 14, wherein the first fluorescent and/or MS-active nucleic acid probe comprises a first fluorescent moiety that fluoresces at a first wavelength and wherein the second fluorescent and/or MS-active nucleic acid probe comprises a second fluorescent moiety that fluoresces at a second wavelength that is different from the first wavelength.

16. A method for detecting a target nucleic acid molecule in a sample containing or suspected of containing said target nucleic acid molecule, the method comprising: (a) contacting the sample with the composition comprising the fluorescent and/or MS-active nucleic acid probe of claim 1, wherein the composition is contacted with the sample under conditions that allow the nucleic-acid-based moiety of the fluorescent and/or MS-active nucleic acid probe to hybridize with the target nucleic acid molecule and form a modified sample containing fluorescent and/or MS-active hybridized moiety; and (c) analyzing the modified sample containing the fluorescent and/or MS-active hybridized moiety using mass spectrometry, using fluorescence spectroscopy, or using both mass spectrometry and fluorescence spectroscopy.

17. The method of claim 16, wherein the nucleic-acid-based moiety contains five or more consecutive nucleotides that are complementary to a sequence of five or more consecutive nucleotides of the target nucleic acid molecule.

18. The method of claim 16, wherein the target nucleic acid molecule comprises RNA, DNA or a mixed oligonucleotide.

19. A method for detecting a target nucleic acid molecule in a sample containing or suspected of containing said a target nucleic acid molecule, the method comprising: (a) contacting the sample with an amine-containing nucleic acid probe that comprises a nucleic-acid-based moiety and a primary or secondary amine under conditions that allow the nucleic-acid-based moiety of the amine-containing nucleic acid probes to hybridize with the target nucleic acid molecule and form a sample that contains a hybridized moiety; (b) contacting the sample that contains the hybridized moiety with a fluorescent and/or MS-active tagging compound that comprises (i) a fluorescent moiety comprising a fluorophore, an MS-active moiety, or both a fluorescent moiety comprising a fluorophore and an MS-active moiety and (ii) a reactive moiety that is reactive with the primary or secondary amine of the amine-containing nucleic acid probe, thereby forming a modified sample that contains a fluorescent and/or MS-active hybridized moiety; and (c) analyzing the sample that contains the fluorescent and/or MS-active hybridized moiety using mass spectrometry, using fluorescence spectroscopy, or using both mass spectrometry and fluorescence spectroscopy.

20. A kit comprising (1) a first fluorescent and/or MS-active nucleic acid probe that comprises (a) a first nucleic-acid-based moiety and (b) a fluorescent moiety comprising a fluorophore, an MS-active moiety, or both a fluorescent moiety and an MS-active moiety, linked to the first nucleic-acid-based moiety and (2) a second fluorescent and/or MS-active nucleic acid probe that comprises (a) a second nucleic-acid-based moiety and (b) a fluorescent moiety comprising a fluorophore, an MS-active moiety, or both a fluorescent moiety and an MS-active moiety, linked to the second nucleic-acid-based moiety.
